(19) United States
(12) Patent Application Publication
(10) Pub. No.: US 2004/0223946 A1
(43) Pub. Date: Nov. 11, 2004

(54) Adhesive treatment for insect and related bites

(75) Inventors: Clark J. Kidd JR., Madison Heights, VA (US); Daniel L. Hedgpeth, Raleigh, NC (US); Upvan Narang, Raleigh, NC (US); Gabriel N. Szabo, Raleigh, NC (US)

Correspondence Address: OLIFF & BERRIDGE, PLC, P.O. BOX 19928, ALEXANDRIA, VA, 22320, US

(73) Assignee: Closure Medical Corporation, Raleigh, NC

(21) Appl. No.: 10/429,050

(22) Filed: May 5, 2003

(51) Int. Cl. A61K 31/765

(52) U.S. Cl. 424/78.27; 424/405

(57) ABSTRACT

A method of treating insect bites includes applying a polymerizable monomer adhesive composition to an area of skin afflicted with an insect bite, optionally with at least one of an additional active agent, and allowing the polymerizable monomer composition to polymerize to form a polymer film over the area of skin.

### BACKGROUND OF THE INVENTION

1. Field of Invention

The present invention relates to treatment and/or amelioration of symptoms of insect and related bites, and particularly relief of the itching sensation associated with such bites. The present invention is thus directed, for example, to insect bites, arachnid bites, mite bites, chigger bites, insect infestations, and the like. More particularly, the present invention relates to methods and compositions for the treatment and/or amelioration of symptoms of such insect and related bites, including treatment and/or amelioration of symptoms such as pain, swelling, infection, itching and the like.

2. Description of Related Art

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and polymers, they have found wide use due to the speed with which they cure, the strength of the resulting bond formed, and their relative ease of use. These characteristics have made the α-cyanoacrylate adhesives the primary choice for numerous applications such as bonding plastics, rubbers, glass, metals, wood, and, more recently, biological tissues.

It is known that monomeric forms of α-cyanoacrylates are extremely reactive, polymerizing rapidly in the presence of even minute amounts of an initiator, including moisture present in the air or on moist surfaces such as animal (including human) tissue. Monomers of α-cyanoacrylates are anionically polymerizable or free radical polymerizable, or polymerizable by zwitterions or ion pairs to form polymers. Once polymerization has been initiated, the cure rate can be very rapid.

Medical applications of 1,1-disubstituted ethylene adhesive compositions include use as an alternate or an adjunct to surgical sutures and/or staples in wound closure, as well as for covering and protecting surface wounds such as lacerations, abrasions, burns, stomatitis, sores, minor cuts and scrapes, and other wounds. When an adhesive is applied to surfaces to be joined, it is usually applied in its monomeric form, and the resultant polymerization gives rise to the desired adhesive bond.

U.S. Pat. Nos. 5,514,371, 5,514,372, 5,575,997, 5,624,669, and 5,582,834 to Leung et al. disclose the addition of a therapeutic agent in a cyanoacrylate composition. The cyanoacrylate adhesive forms a matrix for the therapeutic agent, with the therapeutic agent being released in vivo over time from the matrix during biodegradation of the polymer.

U.S. Pat. No. 5,762,955 to Smith discloses a treatment for healthy, damaged, diseased, or infected biological tissue by applying a bioadhesive coating in conjunction with a medication. The treatment is directed, in part, to treating external biological tissue that may be affected by harmful afflictions such as bruises, burns, dermatological afflictions, infections, gashes, wounds, herpes sores, canker sores, or intra-oral lesions, and skin cancers such as leukemia. Smith further discloses several medications that may be used including corticosteroids, fluoroouracil, obtundants, anesthetics, antibiotics, fungicides, anti-inflammatory agents, antibacterial agents, antiseptic agents, and other medications or combinations of medications used in processes for healing tissue, promoting or preventing blood clotting, destroying cancer cells, palliative treatments and killing of bacteria or viruses.

U.S. Pat. No. 4,880,416 to Horiuchi et al., discloses a dermal bandage of a pre-formed film-like adhesive material for preventing dermally applied ointments, creams, solutions, powders, etc. from falling off, and for delivering drugs, such as anti-fungal agents, to affected parts of the skin. U.S. Pat. Nos. 5,716,607 and 5,716,608, both to Byram et al., disclose the use of cyanoacrylate adhesives to prevent ionization radiation damage to skin. Such damage is prevented by applying the cyanoacrylate polymer to the skin to be protected. U.S. Pat. No. 5,653,769 to Barley, Jr., et al., discloses protecting skin areas from irritation due to contact with artificial devices such as prosthetics, bandages and casts by applying a cyanoacrylate polymer to the desired skin areas that otherwise would be prone to ulceration or irritation by the devices.

U.S. Pat. No. 4,287,177 to Nakashima et al. discloses a protective covering material for forming a film or coat on the skin or wound surface, wherein the film may contain an anti-fungal agent that is controllably released when the composition is placed in contact with the skin. U.S. Pat. Nos. 5,684,042; 5,753,699; 5,762,919; 5,783,177; and 5,811,091 to Greff et al. disclose a cyanoacrylate composition with a compatible anti-fungal agent to form an anti-fungal polymeric cyanoacrylate film to be applied on mammalian skin as wound dressings, wound bandages, surgical incise drapes, wound closure materials and the like.

U.S. Pat. No. 5,160,737 to Friedman et al. discloses a liquid methacrylic acid copolymer composition that contains a release adjusting agent and a pharmacological agent. The composition forms a solid film upon drying, and is capable of accomplishing the sustained release of the pharmacological agent such as to permit its use in the treatment or prevention of dental or dermatological conditions. The composition is described to be useful for treating dermatological conditions such as fungal infections, bacterial infections, viral infections, bums, insect bites, impetigo, tumors, etc.

Insect bites are frequent occurrences in virtually all environments, both indoors and outdoors. Insect bites are thus a common occurrence, especially to those who spend any time outdoors, and particularly in warmer weather. For most individuals, insect bites are nothing more than a nuisance, which cause momentary pain and inflammation, which can subside over a short period of time. However, some insect bites raise higher concerns, since some insects are known to carry disease or bacteria, which can lead to minor or more severe health concerns. For example, mosquitos are well known to carry various diseases, including malaria and West Nile virus, and ticks are known to carry Rocky Mountain spotted fever. In other individuals, who exhibit allergic reactions to insect bites, a single insect bite can pose a serious health concern requiring immediate medical attention. In almost all cases, however, insect bites can result in minor short- or longer-term pain and skin inflammation and irritation. Insect bites typically also cause an itching sensation, which, if the patient scratches the itch too much, can lead to further injury.

As used herein, “insect” or “insects” are used generically to refer not only to the biological species insecta, but also to related species that are commonly referred to, albeit improperly, as “insects” or “bugs.” Thus, for example, “insect” or “insects” are used herein to refer to insecta, arachnids, mites, chiggers, and the like. Similarly, as used herein, “bite” is used generically to refer to an insect bite per se, such as a mosquito bite or wasp, bee or hornet sting, as well as to infestations such as burrowing, or the like, as in the case of ticks.

Physicians commonly prescribe medications in the form of powders, aerosols, liquids or creams for the treatment of insect bites. Such medications are even more commonly obtained by individuals “over-the-counter” for treatment of the same condition.

Often insect bites occur in areas of the skin that are exposed to the air, including the face, arms, legs, and, in warmer weather, back and chest. Such areas are often exposed to higher levels of moisture (such as by perspiration) and/or come into frequent frictional contact with an individual's clothing. As a result of this moisture and/or frictional contact with clothing and the like, topically applied medications can be more easily removed from the site of the insect bite. Moreover, the topically applied medications are more prone to the effects of moisture that is present at the bite site. These difficulties mean that treatment is significantly reduced, because the medication is not held in place for a sufficient time. As a result, the efficacy of the treatment is significantly reduced, and patients must frequently reapply medications so that the bite site receives proper treatment. Furthermore, the inconvenience of bandages due to constant or frequent movement and flexing of the skin, the small surface area usually involved, friction and moisture, make bandages impractical for minimizing frictional contact that occurs at the affected areas of a patient's skin, as well as impractical as a means to hold the medication in place for longer periods of time.

A still further concern in some individuals is the social aspect of the unsightly appearance of inflamed skin areas at the bite site. Accordingly, the visible presence of inflamed skin is highly disfavored. Such individuals thus prefer treatment protocols that are fast and minimally visible.

Accordingly, conventional treatment protocols for insect bites typically involve topical application of lotions and creams, which may optionally be medicated. A problem with such protocols, as described above, is that the lotion or cream may not remain in place for a sufficient length of time to provide effective treatment.

Despite the known use of adhesives, such as described above, such adhesives have not been used in the treatment of insect bites. Instead, the majority of insect bite treatments currently on the market include topically applied medications that have the problems of being easily removed from the application site, and being prone to moisture effects. Therefore, there is a need for an alternative insect bite treatment that remains at the application site for longer periods, that provides fast, effective relief from the symptoms of the insect bite, and that can be either easily removed or maintained in place for longer periods of time without being conspicuously present.

### SUMMARY OF THE INVENTION

The present invention provides methods for treating and/or ameliorating the symptoms of insect bites by applying a monomeric adhesive composition to the affected area. The monomeric adhesive composition of the present invention preferably comprises a polymerizable 1,1-disubstituted ethylene monomer such as a cyanoacrylate monomer, that may optionally include or be accompanied by an additional medicament. The composition reduces moisture and frictional contact with the affected skin area, keeps any active ingredients in contact with the affected skin area for a longer time, provides fast and effective treatment of insect bites, and reduces or eliminates symptoms of insect bites, such as minor pain and itching sensations.

The present invention provides an unexpected treatment composition and method for insect bites, because polymerizable monomers such as 1,1-disubstituted ethylene monomers and cyanoacrylates have not previously been used to treat or prevent such skin conditions. Moreover, while such polymerizable monomers have been variously used on other parts of the body, such as for sealing open wounds, their use for treating insect bites is an entirely new and unexpected use of the materials.

The present treatment for insect bites is advantageous for several reasons. First, because insect bites are common on areas of skin that are prone to moisture or frictional contact, it is often very difficult for the patient to keep a treatment agent in contact with the affected skin area for an extended period of time. This is either because frictional contact with the affected area tends to disturb any applied active agents, or because higher levels of moisture present in the area tend to affect or likewise disturb the applied agent. In addition, because many treatment products and methods are aesthetically non-preferred, especially when used on exposed skin areas, the products are not used to the fullest extent possible to maximize their beneficial effect. The present invention addresses these drawbacks of the prior art, by providing a treatment method and composition that permit improved treatment of insect bites.

Because cyanoacrylates tend to polymerize rapidly to form a relatively robust polymerized film, cyanoacrylates applied to an affected area of a patient's skin can provide fast, effective protection over the affected area and other skin surfaces. By protecting the affected area from moisture and frictional contact, the cyanoacrylate compound can hold any applied active agents in place for a longer period of time and can accordingly significantly increase the time of exposure of an applied medication to the affected area, and ensure more effective treatment.

In addition to forming a stronger barrier to keep moisture and friction away from the affected area while maintaining active agents in contact with the area, cyanoacrylate compounds are also desirable for the treatment of insect bites because of their inherent antipruritic properties. Because the cyanoacrylates form a mechanical barrier over the insect bite, the cyanoacrylate provides itch relief as compared to an exposed insect bite. Itch relief is provided by preventing or ameliorating the itching sensation, which in turn alleviates the perceived need of the patient to scratch the affected area, which in turn could result in tissue damage, infection, and the like. This itch relief can be further improved by incorporating into the cyanoacrylate formulation, or beneath the applied cyanoacrylate film, an additional antipruritic agent.

Further, cyanoacrylate compounds are also desirable for the treatment of insect bites because of their inherent anti-microbial properties. It has been demonstrated in at least some laboratory tests that some cyanoacrylate compounds or formulations provide microbial barrier and anti-microbial properties.

Because of these anti-itch and anti-microbial properties, such cyanoacrylate compounds and formulations may be especially desirable for treating insect bites, even without the introduction or pre-application of other active agents or medicaments. Moreover, the properties of such cyanoacrylate compounds and formulations may also be beneficial in instances where an active agent is applied before, or together with, the cyanoacrylate, because the cyanoacrylate can continue to provide its intended effects even after the applied agent has been completely absorbed or used up.

Cyanoacrylate compounds are also useful as carriers or delivery agents for anti-pruritic materials or other active materials. And as a result, it enhances the treatment of the affected area by controllably releasing the agent to the affected area.

### DETAILED DESCRIPTION OF PREFERRED EMBODIMENTS

This invention is directed to methods of and compositions for treating insect bites. In its method aspects, the present invention is directed to methods comprising applying a monomeric adhesive composition, comprising an α-cyanoacrylate, with or without optional additives, to an afflicted area of skin. In its composition aspects, the present invention is directed to compositions comprising a monomeric adhesive composition and one or more additives, which composition is useful in the treatment and/or amelioration of symptoms of insect bites.

According to embodiments of the present invention, the adhesive composition can be applied alone, or it can be applied subsequent to or concurrent with the application of a separate suitable active agent, such as an anti-pruritic agent, antiseptic, pain reliever, or the like. Furthermore, in embodiments, the monomeric adhesive composition can itself include an active agent in addition to polymerizable monomer.

According to the present invention, “treat” (or other forms of the word such as “treating” and “treatment”) refers to employment of the methods and/or compositions against an existing insect bite or infestation (i.e., a site where an insect such as a tick has burrowed into the skin). “Treat” thus encompasses both amelioration of effects of insect bites, such as by reducing swelling, inflammation, irritation, itching, pain and the like, and active reduction of the conditions such as by speeding or assisting healing and killing any bacterial growth that may be present in the afflicted area. Treatment is thus distinguished from prevention, which involves unaffected areas of skin in the absence of an insect bite.

In embodiments of the invention, an adhesive composition can be used alone for treating insect bites. In such embodiments, a patient or care-provider simply applies the composition to an area of the skin afflicted with the insect bite. The composition then is permitted to polymerize to form a robust polymeric coating that effectively covers and protects the affected or applied area of the patient's skin. The composition may inhibit or kill microbes, and the robust polymeric coating that the composition forms protects the area from frictional contact with clothing or other skin surfaces so that prolonged treatment or prevention can be achieved.

Preferably, the composition is applied in a sufficient amount to entirely cover the desired area, which generally would correspond to an affected area where the insect bite is present. In embodiments, the composition covers an additional area around the desired area, for example to cover an area of skin that is inflamed or susceptible to such sensations as pain or itching.

When applied, the compositions and methods of the present invention may be used to effectively treat the symptoms of an insect bite. For example, the applied composition, with or without suitable active ingredients or other additives, can reduce inflammation and swelling, can reduce sensations of pain or itchiness, and can help kill any bacteria that may have entered the bite site form the insect or that may have subsequently entered the bite site. The compositions and methods of the present invention also provide an adherent coating over the bite site, which may help to reduce irritation of the bite site.

According to the present invention, the adhesive composition is preferably permitted to substantially or fully polymerize to form a polymer film before the treated area is permitted to contact other surfaces, such as articles of clothing. Thus, for example, when the composition is applied to areas of skin, any adjoining skin surfaces are preferably kept separated from each other until the composition has substantially or fully polymerized, to prevent bonding the skin areas together. Likewise, for example where the composition is applied to areas of skin that lie beneath clothing, such as on the chest or back, the skin surfaces are preferably kept separated from the clothing until the composition has substantially or fully polymerized, to prevent bonding the skin area and clothing together.

Once applied and set, the polymerized composition is preferably retained on the application site for a time sufficient to permit treatment and/or amelioration of symptoms of insect bites. Thus, for example, the polymerized composition is preferably retained on the application site for a period of at least one or two hours or more, more preferably for at least four or six hours or more, and even more preferably for at least eight, sixteen or 24 hours or more. Once applied, the polymerized composition can be retained on the application site for as long as desired, and generally until the polymerized composition is “sloughed off” the skin as the outer layers of the skin normally progress. Thus, for example, the polymerized composition can be retained on the application site for up to 5, 6 or 7 days, or even more. In embodiments, for example, the polymerized composition is preferably retained on the application site for from about 1 or 2 to about 4 or 5 days, to provide adequate results. Of course, shorter or longer application times can be used, as appropriate based on the particular polymerized composition and/or insect bite.

In embodiments, the polymerized composition is retained on the application site for a given period of time, and can be either prematurely removed or naturally removed by allowing the polymerized composition to slough off of the application site. Thus, for example, although the polymerized composition would typically adhere to the application site on skin for about 5 to about 7 days before naturally sloughing off, it is possible in embodiments that the polymerized composition can be removed prior to such natural sloughing off. Such removal can be accomplished, for example, by removing the polymerized composition by using such measures as soap and water or other conventional skin cleansers.

In terms of removal of the polymerized formulation, suitable biocompatible remover compositions are disclosed in, for example, U.S. patent application Ser. No. 09/962,268, filed Sep. 26, 2001, the entire disclosure of which is incorporated herein by reference. Alternatively, it is possible to formulate the polymerizable monomer composition itself to permit easy removal using oil-based cleansers, such as mineral oil or baby oil, and/or mechanical rubbing, such as with a wash cloth.

According to the present invention, the composition and method can be practiced to treat insect bites in any of its various stages. Thus, for example, the methods and compositions of the present invention can be used to treat insect bites immediately after the bite occurs, such as within about 1, about 5 or about 10 minutes after the insect bite occurs, or can be used to treat insect bites subsequent to the bite but prior to complete healing, such as when symptoms such as itching or the like occur.

In further embodiments of the present invention, the adhesive composition can be applied over a medicament for treating the insect bite. The medicament in this embodiment is not particularly limited, and can include any of the available medicaments or agents for the skin conditions that occur as a result of insect bites. The medicament can also be, or comprise, any suitable anti-inflammatory or anesthetic, anti-pruritic agent, other active agents, or other treatment agents for insect bites, as described below. All such agents are generally referred to herein as “active agents” unless otherwise specified. In this embodiment, the medicament can be first applied to the affected or desired area, followed by application of a polymerizable adhesive composition. The medicament can be in any suitable form, including liquid, solid, powder, cream or the like, and can include only a medicament or can include other suitable additives such as diluents, carriers or the like. Where the medicament is in a liquid or a semi-liquid form, it is preferred that the medicament be permitted to dry, substantially or completely, prior to application of the adhesive composition. However, the adhesive composition can also be immediately applied over the applied medicament, or can be applied prior to drying of the medicament, if desired.

In embodiments of the present invention, an appropriate, preferably monomer-compatible, active agent can be mixed with the polymerizable adhesive composition and a resultant composition applied to the affected or desired area. In this embodiment, the active agent can be mixed with the polymerizable adhesive composition during manufacture (i.e. prior to packaging the materials), or immediately prior to use. However, the present invention is not limited to such embodiments. Thus, for example, the agent need not be monomer-compatible. In these embodiments, the agent can be mixed or combined with the polymerizable adhesive composition, usually just prior to application, and a resultant composition applied to the affected or desired area

In further embodiments of the present invention the active agent may also serve as a polymerization initiator or a stabilizer. Thus, the agent can provide not only a biological activity, but a chemical one as well.

Active agents that also serve as polymerization initiators can initiate and/or accelerate the polymerization of the composition when applied to an affected or desired area of skin. Accelerated polymerization reduces the waiting time necessary after application, and makes the composition more convenient to apply. Suitable active agents that can also serve as initiators include, but are not limited to, certain acidic and quaternary ammonium compounds. In embodiments where the agent also acts as a polymerization initiator or rate modifier, the present invention provides the additional advantage of not requiring that a further, separate polymerization initiator or rate modifier be used. Furthermore, in these embodiments, the agent is preferably located in a non-contacting relationship with the adhesive composition prior to use, so that premature polymerization of the adhesive composition does not occur.

Active agents that also serve as stabilizers can extend the useful life of the composition. By increasing the useful life of the composition, the composition can be stored and packaged for longer periods of time without the risk of premature polymerization. Suitable active agents that can also serve as stabilizers can include, but are not limited to, certain acidic and phenolic compounds. In embodiments where the agent also acts as a stabilizer for the adhesive composition, the present invention provides the additional advantage of not requiring that a further, separate stabilizer be used. Furthermore, in these embodiments, the agent is preferably located in a contacting relationship with the adhesive composition, such as being mixed with the adhesive composition, prior to use.

When the additives are mixed with the monomer composition during storage, it is preferred that the mixture exhibit a sufficiently long shelf-life to permit economical commercial distribution of the mixture. Thus, for example, the mixture should exhibit a shelf-life, as measured at room temperature and moderate humidity (about 60% relative humidity), of at least about one year, and preferably at least about two or even at least about three years. Where the additive and monomer are not mixed during storage, it is still preferred that the separate components exhibit similar shelf-lives to those of a mixed composition. As used herein, “shelf-life” refers to the amount of time the container and composition therein can be held at approximately room temperature (21-25° C.) without degradation of the composition and/or container occurring to the extent that the composition and container cannot be used in the manner and for the purpose for which they were intended. Thus, while some degradation to either or both of the composition and container can occur, it must not be to such an extent that the composition and/or container is no longer useable. Shelf-life can thus be limited by physical or aesthetic changes to the containers or products contained therein, by chemical reactions occurring within the composition being stored, by chemical reactions between the container and the composition being stored, by degradation of the container itself, and the like.

Although a mixture of active agent and polymerizable monomer, according to the present invention, is not limited to a specific ratio of agent to polymerizable monomer, the agent is preferably present in an effective amount, preferably a therapeutically effective amount for treating the insect bite or particular symptom thereof.

When mixed or combined immediately prior to use, the active agent can be mixed with the polymerizable monomer composition in a suitable container and thereafter applied. Alternatively, mixing can be conducted during the application process, for example by using an applicator that is loaded with agent, which thereby mixes the agent with the adhesive composition during application.

Suitable active agents include, but are not limited to, known agents such as anti-pruritic agents, analgesics, anesthetics, and counter-irritants (all of which may generally be referred to as anti-itch agents). Examples of suitable anti-pruritic agents include, but are not limited to, -amine or -caine type local anesthetics, such as benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, and tetracaine hydrochloride; alcohols and ketones, such as benzyl alcohol, camphor, camphorated metacresol, juniper tar, menthol, phenol, phenolate sodium, and resorcinol; antihistamines, such as hydrocortisone and hydrocortisone acetate; mixtures thereof, and the like. Examples of suitable counter-irritant active agents include, but are not limited to, irritants that produce redness, such as allyl isothiocyanate, strong ammonia solution, methyl salicylate, and turpentine oil; irritants that produce cooling sensation, such as camphor and menthol; irritants that produce vasodilation, such as histamine dihydrochloride and methyl nicotinate; irritants that do not produce redness, such as capsaicin, capsicum, and capsicum oleoresin; mixtures thereof, and the like. Other suitable additives include, but are not limited to, chlorobutanol, crotamiton, cyproheptadine, dichlorisone, doxepin, halometasone, 3-hydroxycamphor, mesulfen, methdilazine, oil of bitter almond, polidocanol, spirit of camphor, rectified tar oil, thenaldine, tolpropamine, trimeprazine, mixtures thereof, and the like. Mixtures of any two or more of the foregoing can also be used, as desired.

Suitable anti-microbial agents include, but are not limited to, known agents such as parabens, cresols, azoles, allylamines, pollyenes, acidics, mercurials, quaternary ammonium compounds, other agents, non-polymer-stabilized compounds, i.e., that are not complexed with or otherwise part of a polymer species, mixtures thereof, and the like. Such anti-microbial agents should preferably be present in a therapeutically effective amount, particularly in cases where higher amounts may otherwise be toxic to the patient. Suitable such agents are disclosed in, for example, U.S. patent application Ser. No. 09/898,092, filed Jul. 5, 2001, the entire disclosure of which is incorporated herein by reference, and are designated therein as “anti-fungal agents.”

In addition, in embodiments where monomer additives including, but not limited to those listed above, are insoluble with the monomer composition and/or that would cause premature polymerization of the monomer, the additive can be applied to a skin area before applying the monomer composition. In such embodiments, the additive and the monomer composition can be provided, for example, in separate packages in a kit.

In other embodiments, where such additives are soluble with the monomer composition and/or would not cause premature polymerization of the monomer, the additives can be combined with the monomer composition during manufacture of the composition. Moreover, in cases where the additive is soluble with the monomer composition, the additive can be applied before the monomer composition is applied, it can be pre-mixed with and applied together with the monomer composition, it can be mixed together with the monomer composition immediately before application, or it can even be applied after the monomer composition has been applied. As a result, in cases where a soluble additive is to be applied, the additive and the composition can be provided in a kit where the additive and the monomer composition are pre-mixed, or the additive and the monomer composition can be provided separately to be applied separately or mixed together immediately prior to, during, or after application.

Although a mixture of additive and monomer composition according to the present invention is not limited to a specific ratio of additive to polymerizable monomer, the additive is preferably present in an effective amount, and preferably in a therapeutically effective amount.

When mixed immediately prior to use, the additive can be mixed with the polymerizable monomer composition in a suitable container and thereafter applied. Alternatively, mixing can be conducted during the application process, for example by using an applicator loaded with the additive, which thereby mixes the additive with the adhesive composition during application.

In embodiments, the monomer composition and/or its packaging can be sterilized. However, sterilization is by no means required, particularly in view of the fact that most commercially available products for the treatment of insect bites are not sterilized. Furthermore, whether or not the composition and container is sterilized, the composition can further include one or more suitable preservative, as described below.

Sterilization of the monomer composition and/or its packaging can be accomplished by techniques known to the skilled artisan, and is preferably accomplished by methods including, but not limited to, chemical, physical, and/or irradiation methods. Examples of chemical methods include, but are not limited to, exposure to ethylene oxide or hydrogen peroxide vapor. Examples of physical methods include, but are not limited to, sterilization by heat (dry or moist) or retort canning. Examples of irradiation methods include, but are not limited to, gamma irradiation, electron beam irradiation, and microwave irradiation. A preferred method is electron beam irradiation, as described in U.S. Pat. No. 6,143,805, the entire disclosure of which is incorporated herein by reference. The composition should also show low levels of toxicity to living tissue during its useful life. In preferred embodiments of the present invention, the composition is sterilized to provide a Sterility Assurance Level (SAL) of at least 10⁻³. In embodiments, the Sterility Assurance Level may be at least 10⁻⁴, or may be at least 10⁻⁵, or may be at least 10⁻⁶.

The monomer (including prepolymeric) adhesive composition may include one or more polymerizable monomers. Preferred monomers that may be used in this invention are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687, 5,928,611 and 6,183,593, U.S. patent application Ser. No. 09/430,177, filed on Oct. 29, 1999, and U.S. Pat. No. 6,183,593, which are hereby incorporated in their entirety by reference herein.

Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 3 to about 8 carbon atoms.

The α-cyanoacrylates of the present invention can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

Preferred α-cyanoacrylate monomers used in this invention include methyl cyanoacrylate, ethyl cyanoacrylate, n-butyl cyanoacrylate, 2-octyl cyanoacrylate, methoxyethyl cyanoacrylate, ethoxyethyl cyanoacrylate, dodecyl cyanoacrylate, 2-ethylhexyl cyanoacrylate, butyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, hexyl cyanoacrylate, or dodecylcyanoacrylate.

Other suitable cyanoacrylates for use in the present invention also include, but are not limited to, alkyl ester cyanoacrylate monomers such as those having the formula

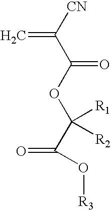

wherein R₁ and R₂ are, independently H, a straight, branched or cyclic alkyl, or are combined together in a cyclic alkyl group, and R₃ is a straight, branched or cyclic alkyl group. Preferably, R₁ is H or a C₁, C₂ or C₃ alkyl group, such as methyl or ethyl; R₂ is H or a C₁, C₂ or C₃ alkyl group, such as methyl or ethyl; and R₃ is a C₁-C₁₆ alkyl group, more preferably a C₁-C₁₀ alkyl group, such as methyl, ethyl, propyl, butyl, pentyl, hexyl, heptyl, octyl, nonyl or decyl, and even more preferably a C₂, C₃ or C₄ alkyl group. Such alkyl ester cyanoacrylates and other suitable monomers are disclosed in, for example, U.S. patent applications Ser. Nos. 09/630,437, filed Aug. 2, 2000, and 09/919,877, filed Aug. 2, 2001, the entire disclosures of which are incorporated herein by reference.

Examples of preferred alkyl ester cyanoacrylates include, but are not limited to, butyl lactoyl cyanoacrylate (BLCA), butyl glycoloyl cyanoacrylate (BGCA), ethyl lactoyl cyanoacrylate (ELCA), and ethyl glycoloyl cyanoacrylate (EGCA). BLCA may be represented by the above formula, wherein R₁ is H, R₂ is methyl and R₃ is butyl. BGCA may be represented by the above formula, wherein R₁ is H, R₂ is H and R₃ is butyl. ELCA may be represented by the above formula, wherein R is H, R₂ is methyl and R₃ is ethyl. EGCA may be represented by the above formula, wherein R₁ is H, R₂ is H and R₃ is ethyl.

The composition may optionally also include at least one other plasticizing agent that assists in imparting flexibility to the polymer formed from the monomer. The plasticizing agent preferably contains little or no moisture and should not significantly affect the stability or polymerization of the monomer. Examples of suitable plasticizers include but are not limited to silica particles, tributyl citrate, acetyl tri-n-butyl citrate (ATBC), polymethylmethacrylate, silicone oils, siloxanes and others as listed in U.S. Pat. No. 6,183,593, the disclosure of which is incorporated in its entirety by reference herein. Specific examples of the silicone oils and siloxanes include, for example, but are not limited to, polydimethylsiloxane, hexadimethylsilazane.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Examples of suitable thixotropic agents and thickeners are disclosed in, for example, U.S. Pat. No. 4,720,513, and U.S. Pat. No. 6,310,166, the disclosures of which are hereby incorporated in their entireties by reference herein.

The composition may optionally also include thickeners. Suitable thickeners may include poly (2-ethylhexy methacrylate), poly(2-ethylhexyl acrylate) and others as listed in U.S. Pat. No. 6,183,593, the disclosure of which is incorporated by reference herein in its entirety.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties by reference herein.

The composition may optionally also include one or more stabilizers, preferably both at least one anionic vapor phase stabilizer and at least one anionic liquid phase stabilizer. These stabilizing agents may inhibit premature polymerization. Suitable stabilizers may include those listed in U.S. Pat. No. 6,183,593, the disclosure of which is incorporated by reference herein in its entirety. Furthermore, certain stabilizers may also function as active agents, such as, for example, various acidic agents, as identified above.

The stability, and thus the shelf-life, of some monomeric adhesive compositions can be further enhanced and extended through careful regulation of the packaging. Treated (e.g., fluorinated polymer) packaging such as that disclosed in copending U.S. patent application Ser. No. 09/430,289, filed Oct. 29, 1999, which is hereby incorporated by reference herein in its entirety, is preferred and may reduce the amount of stabilizer that is combined into the composition. As mentioned above, certain stabilizers including, but not limited to, certain acidics can also function as active agents. In this case, the amount of the active agent/stabilizer material is either not reduced below a level to provide the desired effect, or a further active agent/non-stabilizing agent is added to ensure that the desired effect is provided.

The compositions may also include pH modifiers to control the rate of degradation of the resulting polymer, as disclosed in U.S. Pat. No. 6,143,352, the entire disclosure of which is hereby incorporated by reference herein in its entirety.

Compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds useful in this invention include sulfites; bisulfites; mixtures of sulfites and bisulfites, etc. Additional examples of formaldehyde scavenger compounds useful in this invention and methods for their implementation can be found in U.S. Pat. Nos. 5,328,687, 5,514,371, 5,514,372, 5,575,997, 5,582,834 and 5,624,669, all to Leung et al., which are incorporated herein by reference in their entireties.

To improve the cohesive strength of adhesives. formed from the compositions of this invention, difunctional monomeric cross-linking agents may be added to the monomer compositions of this invention. Such crosslinking agents are known. U.S. Pat. No. 3,940,362 to Overhults, which is hereby incorporated herein in its entirety by reference, discloses exemplary cross-linking agents.

The compositions of this invention may further contain fibrous reinforcement and colorants such as dyes, pigments, and pigment dyes. Examples of suitable fibrous reinforcement include PGA microfibrils, collagen microfibrils, and others as described in U.S. Pat. No. 6,183,593, the disclosure of which is incorporated by reference herein in its entirety.

The polymerizable compositions useful in the present invention may also further contain one or more preservatives, for prolonging the storage life of the composition. Suitable preservatives, and methods for selecting them and incorporating them into adhesive compositions, are disclosed in U.S. patent application Ser. No. 09/430,180, the entire disclosure of which is incorporated herein by reference. Such preservatives can be in addition to any antimicrobial agent that may or may not be added to the composition, as described above. Such preservatives can be included irrespective of whether the composition and containers are sterilized.

In embodiments of the present invention, the composition and/or its applicator may contain materials such as a polymerization initiator, accelerator, rate-modifier, and/or cross-linking agent for initiating polymerization and/or cross-linking of the polymerizable monomer material. Suitable materials and applicators and packaging systems are disclosed in U.S. Pat. Nos. 5,928,611, 6,352,704 and 6,455,064 and U.S. patent applications Ser. Nos. 09/430,177, 09/430,289, 09/430,290, and 09/430,180 filed Oct. 29, 1999; 09/385,030 filed Aug. 30, 1999; and 09/176,889 filed Oct. 22, 1998; the entire disclosures of which are incorporated herein by reference.

According to the present invention, any suitable applicator can be used to apply the composition to the affected areas of skin. Suitable applicators and packaging systems are disclosed in, for example, U.S. Pat. Nos. 5,928,611, 6,352,704 and 6,455,064 and U.S. patent applications Ser. Nos. 09/430,177, 09/430,289, 09/430,290, and 09/430,180 filed Oct. 29, 1999; 09/385,030 filed Aug. 30, 1999; 09/176,889 filed Oct. 22, 1998, and 09/898,006 filed Jul. 5, 2001; the entire disclosures of which are incorporated herein by reference.

### EXAMPLES

### Example 1

A 2-octyl cyanoacrylate monomer composition is prepared by adding 1 wt % menthol to 2 mL of 2-octyl cyanoacrylate monomer. The mixture is stirred.

The characteristics of the composition are observed at about one minute after preparation and later at least twenty-four hours after preparation. The results of the observations show that the solution remains clear, indicating that menthol is soluble in the monomer and does not cause premature polymerization.

The composition is then applied to areas of skin affected by an insect bite, either immediately after the bite occurs, or at any time prior to complete healing of the bite site. The monomer composition polymerizes in under one minute, resulting in a polymerized film of material covering the affected area. The polymerized film is left in place for a desired period of time, as described above.

### Example 2

A 2-octyl cyanoacrylate monomer composition is formed and tested in the same manner as Example 1, above, except that the 1 wt % menthol is replaced by 3 wt % camphor. The same observations and results are obtained as reported in Example 1 above.

While the invention has been described with reference to preferred embodiments, the invention is not limited to the specific examples given, and other embodiments and modifications can be made by those skilled in the art without departing from the spirit and scope of the invention.

1. A method of treating an insect bite, comprising:
a) applying an adhesive composition comprising a polymerizable α-cyanoacrylate monomer to an area of skin that is afflicted with an insect bite; and
b) allowing said polymerizable monomer to polymerize to form a polymer film over said area of skin.

2. The method of claim 1, wherein said insect bite is an insecta bite.

3. The method of claim 1, wherein said insect bite is an arachnid bite.

4. The method of claim 1, wherein said insect bite is an insect infestation.

5. The method of claim 1, wherein said area of skin exhibits a symptom selected from the group consisting of inflammation, irritation, itching, pain, and bacterial infection.

6. The method of claim 1, wherein said composition is applied within about 10 minutes after the insect bite occurs.

7. The method of claim 1, wherein said composition is applied more than about 10 minutes after the insect bite occurs.

8. The method of claim 1, wherein said polymerizable monomer comprises at least one member selected from the group consisting of ethyl cyanoacrylate, butyl cyanoacrylate, and 2-octyl cyanoacrylate.

9. The method of claim 1, further comprising combining at least one agent selected from the group consisting of active agents with the polymerizable monomer composition, wherein the at least one agent serves as a polymerization initiator for said polymerizable monomer composition.

10. The method of claim 1, wherein said composition further comprises at least one stabilizing agent for said polymerizable monomer.

11. The method of claim 10, wherein said stabilizing agent is also an active agent.

12. The method of claim 1, wherein said composition comprises at least one plasticizer.

13. The method of claim 12, wherein the plasticizer comprises at least one member selected from the group consisting of silica particles, tributyl citrate, acetyl tributyl citrate, polymethylmethacrylate, silicone oils and siloxanes.

14. The method of claim 1, wherein the composition further comprises at least one active agent.

15. The method of claim 14, wherein the active agent is an anti-itch agent.

16. The method of claim 14, wherein the active agent is selected from the group consisting of anti-pruritic agents, analgesics, anesthetics, and counter-irritants.

17. The method of claim 14, wherein the active agent is selected from the group consisting of benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium, resorcinol, hydrocortisone, hydrocortisone acetate, allyl isothiocyanate, strong ammonia solution, methyl salicylate, turpentine oil, histamine dihydrochloride, methyl nicotinate, capsaicin, capsicum, capsicum oleoresin, chlorobutanol, crotamiton, cyproheptadine, dichlorisone, doxepin, halometasone, 3-hydroxycamphor, mesulfen, methdilazine, oil of bitter almond, polidocanol, spirit of camphor, rectified tar oil, thenaldine, tolpropamine, trimeprazine, and mixtures thereof.

18. The method of claim 12, wherein the at least one agent is mixed with the polymerizable monomer composition immediately prior to applying the polymerizable monomer composition to the area of skin.

19. The method of claim 12, wherein the at least one agent is mixed with the polymerizable monomer composition during manufacture of the polymerizable monomer composition.

20. The method of claim 1, wherein said composition has a Sterility Assurance Level (SAL) of 10⁻³-10⁻⁶.

21. The method of claim 1, further comprising applying at least one active agent to the area of skin before applying the adhesive composition.

22. The method of claim 21, further comprising allowing the at least one applied agent to substantially dry before applying the adhesive composition.

23. The method of claim 21, wherein the active agent is an anti-itch agent.

24. The method of claim 21, wherein the active agent is selected from the group consisting of anti-pruritic agents, analgesics, anesthetics, and counter-irritants.

25. The method of claim 21, wherein the active agent is selected from the group consisting of benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium, resorcinol, hydrocortisone, hydrocortisone acetate, allyl isothiocyanate, strong ammonia solution, methyl salicylate, turpentine oil, histamine dihydrochloride, methyl nicotinate, capsaicin, capsicum, capsicum oleoresin, chlorobutanol, crotamiton, cyproheptadine, dichlorisone, doxepin, halometasone, 3-hydroxycamphor, mesulfen, methdilazine, oil of bitter almond, polidocanol, spirit of camphor, rectified tar oil, thenaldine, tolpropamine, trimeprazine, and mixtures thereof.

26. The method of claim 1, wherein said adhesive composition is applied directly to said area of skin, and said adhesive composition does not include an anti-itch agent.

27. The method of claim 26, wherein said polymer film has anti-itch effects at said area of skin.

28. A method of treating an insect bite, the method comprising the steps of:
a. applying at least one active agent to an area of skin that is afflicted with an insect bite;
b. applying a polymerizable monomer composition to said area of skin over the at least one applied agent, wherein said composition comprises a polymerizable α-cyanoacrylate monomer; and
c. allowing said polymerizable monomer composition to polymerize to form a polymer film over said area of skin and said at least one agent.

29. A method of treating insect bites, the method comprising:
a. combining a polymerizable monomer composition comprising a polymerizable α-cyanoacrylate monomer and at least one active agent to form a mixture;
b. applying said mixture to an area of skin that is afflicted with an insect bite; and
c. allowing said mixture to polymerize to form a polymer film over said area of skin.

30. A composition, comprising:
a polymerizable 1,1-disubstituted ethylene monomer, and
an anti-pruritic agent present in an effective amount to reduce itching associated with an insect bite.

31. The composition of claim 30, wherein the anti-pruritic agent is selected from the group consisting of benzocaine, butamben picrate, dibucaine, dibucaine hydrochloride, dimethisoquin hydrochloride, dyclonine hydrochloride, lidocaine, lidocaine hydrochloride, pramoxine hydrochloride, tetracaine, tetracaine hydrochloride, benzyl alcohol, camphor, camphorated metacresol, juniper tar, menthol, phenol, phenolate sodium, resorcinol, hydrocortisone, hydrocortisone acetate, allyl isothiocyanate, strong ammonia solution, methyl salicylate, turpentine oil, histamine dihydrochloride, methyl nicotinate, capsaicin, capsicum, capsicum oleoresin, chlorobutanol, crotamiton, cyproheptadine, dichlorisone, doxepin, halometasone, 3-hydroxycamphor, mesulfen, methdilazine, oil of bitter almond, polidocanol, spirit of camphor, rectified tar oil, thenaldine, tolpropamine, trimeprazine, and mixtures thereof.

32. A composition, comprising:
a polymerizable 1,1-disubstituted ethylene monomer, and
camphor.

33. The composition of claim 32, wherein said polymerizable 1,1-disubstituted ethylene monomer is a polymerizable α-cyanoacrylate monomer.

34. The composition of claim 32, wherein said polymerizable 1,1-disubstituted ethylene monomer comprises at least one member selected from the group consisting of ethyl cyanoacrylate, butyl cyanoacrylate, and 2-octyl cyanoacrylate.

35. The composition of claim 32, wherein said camphor is present in an effective amount to reduce pain or itching.

36. The composition of claim 32, wherein said camphor is present in an effective amount to reduce itching.

37. A method of treating skin, comprising:
a) applying the adhesive composition of claim 32 to an area of skin; and
b) allowing said polymerizable monomer to polymerize to form a polymer film over said area of skin.

38. The method of claim 37, wherein said polymerizable 1,1-disubstituted ethylene monomer is a polymerizable α-cyanoacrylate monomer.

39. The method of claim 37, wherein said polymerizable 1,1-disubstituted ethylene monomer comprises at least one member selected from the group consisting of ethyl cyanoacrylate, butyl cyanoacrylate, and 2-octyl cyanoacrylate.

40. The method of claim 37, wherein said camphor is present in an effective amount to reduce pain or itching.

41. The method of claim 37, wherein said camphor is present in an effective amount to reduce itching.
